(19) United States
(12) Patent Application Publication
(10) Pub. No.: US 2007/0293670 A1
(43) Pub. Date: Dec. 20, 2007

(54) Pyrrolopyrimidine and Pyrrolopyridine Derivatives Substituted with Tetrahydropyridine as Crf Antagonists

(75) Inventors: Atsuro Nakazato, Tokyo (JP); Taketoshi Okubo, Tokyo (JP); Dai Nozawa, Tokyo (JP); Ludo E.J. Kennis, Beerse (BE); Marcel F. L. De Bruyn, Beerse (BE)

Correspondence Address: SUGHRUE MION, PLLC, 2100 PENNSYLVANIA AVENUE, N.W., SUITE 800, WASHINGTON, DC, 20037, US

(73) Assignee: TAISHO PHARMACEUTICAL CO., LTD., Tokyo (JP)

(21) Appl. No.: 11/630,041

(22) PCT Filed: Jun. 24, 2005
(86) PCT No.: PCT/JP05/12141

§ 371 Date: Jul. 23, 2007

(30) Foreign Application Priority Data
Jun. 25, 2004 (JP) 2004-188129

(51) Int. Cl. C07D 487/04

(52) U.S. Cl. 544/280; 546/113

(57) ABSTRACT

[PROBLEM TO BE SOLVED]An object of the present invention is to provide an antagonist against CRF receptors which is effective as a therapeutic or prophylactic agent for diseases in which CRF is considered to be involved, such as depression, anxiety, Alzheimer's disease, Parkinson's disease, Huntington's chorea, eating disorder, hypertension, gastrointestinal diseases, drug dependence, cerebral infarction, cerebral ischemia, cerebral edema, cephalic external wound, inflammation, immunity-related diseases, alopecia, irritable bowel syndrome, sleep disorders, epilepsy, dermatitides, schizophrenia, pain, etc.

[SOLUTION]A pyrrolopyrimidine or pyrrolopyridine derivative substituted with tetrahydropyridine represented by the following formula [I]:

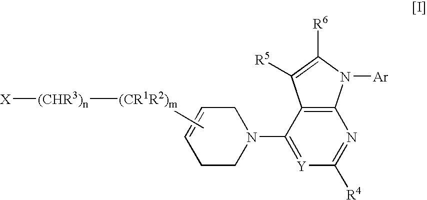

has a high affinity for CRF receptors and is effective against diseases in which CRF is considered to be involved.

### DETAILED DESCRIPTION OF THE INVENTION

1. Technical Field

The present invention relates to a therapeutic agent for diseases in which corticotropin releasing factor (CRF) is considered to be involved, such as depression, anxiety, Alzheimer's disease, Parkinson's disease, Huntington's chorea, eating disorder, hypertension, gastrointestinal diseases, drug dependence, cerebral infarction, cerebral ischemia, cerebral edema, cephalic external wound, inflammation, immunity-related diseases, alopecia, irritable bowel syndrome, sleep disorders, epilepsy, dermatitides, schizophrenia, pain, etc.

2. Description of the Prior Art

CRF is a hormone comprising 41 amino acids (Science, 213, 1394-1397, 1981; and J. Neurosci., 7, 88-100, 1987), and it is suggested that CRF plays a core role in biological reactions against stresses (Cell. Mol. Neurobiol., 14, 579-588, 1994; Endocrinol., 132, 723-728, 1994; and Neuroendocrinol. 61, 445-452, 1995). For CRF, there are the following two paths: a path by which CRF acts on peripheral immune system or sympathetic nervous system through hypothalamus-pituitary-adrenal system, and a path by which CRF functions as a neurotransmitter in central nervous system (in Corticotropin Releasing Factor: Basic and Clinical Studies of a Neuropeptide, pp. 29-52, 1990). Intraventricular administration of CRF to hypophysectomized rats and normal rats causes an anxiety-like symptom in both types of rats (Pharmacol. Rev., 43, 425-473, 1991; and Brain Res. Rev., 15, 71-100, 1990). That is, there are suggested the participation of CRF in hypothalamus-pituitary-adrenal system and the pathway by which CRF functions as a neurotransmitter in central nervous system.

The review by Owens and Nemeroff in 1991 summarizes diseases in which CRF is involved (Pharmacol. Rev., 43, 425-474, 1991). That is, CRF is involved in depression, anxiety, Alzheimer's disease, Parkinson's disease, Huntington's chorea, eating disorder, hypertension, gastrointestinal diseases, drug dependence, inflammation, immunity-related diseases, etc. It has recently been reported that CRF is involved also in epilepsy, cerebral infarction, cerebral ischemia, cerebral edema; and cephalic external wound (Brain Res. 545, 339-342, 1991; Ann. Neurol. 31, 48-498, 1992; Dev. Brain Res. 91, 245-251, 1996; and Brain Res. 744, 166-170, 1997). Accordingly, antagonists against CRF receptors are useful as therapeutic agents for the diseases described above.

WO04/058767, WO02/002549 and WO00/053604 disclose pyrrolopyridine and pyrrolopyrimidine derivatives as CRF receptor antagonists. However, none disclose the compounds provided in the present invention.

### Problem(S) to be Solved by Invention

An object of the present invention is to provide an antagonist against CRF receptors which is effective as a therapeutic or prophylactic agent for diseases in which CRF is considered to be involved, such as depression, anxiety, Alzheimer's disease, Parkinson's disease, Huntington's chorea, eating disorder, hypertension, gastrointestinal diseases, drug dependence, cerebral infarction, cerebral ischemia, cerebral edema, cephalic external wound, inflammation, immunity-related diseases, alopecia, irritable bowel syndrome, sleep disorders, epilepsy, dermatitides, schizophrenia, pain, etc.

### Means for Solving Problem

The present inventors earnestly investigated pyrrolopyrimidine and pyrrolopyridine derivatives substituted with tetrahydropyridine that have a high affinity for CRF receptors, whereby the present invention has been accomplished.

The present invention is pyrrolopyrimidine and pyrrolopyridine derivatives substituted with tetrahydropyridine explained below.

A pyrrolopyrimidine or pyrrolopyridine derivative substituted with tetrahydropyridine represented by the following formula [I]: 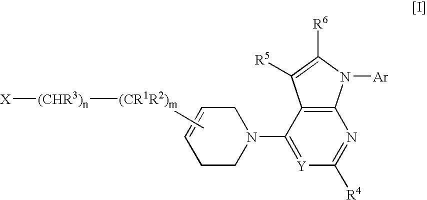
(wherein the tetrahydropyridine is represented by the following formula [II]:

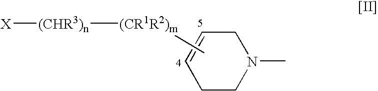

in which the tetrahydropyridine ring is substituted with a group represented by —(CR¹R²)ₘ—(CHR³)ₙ—X at the 4-position or 5-position of the tetrahydropyridine ring;

X is hydroxy, cyano, —CO₂R⁷ or —CONR^{7a}R^{7b};

Y is N or CR⁸;

with the proviso that when Y is CR⁸, then X is hydroxy;

R¹ is hydrogen, hydroxy, C₁₋₅alkyl, C₁₋₅alkoxy-C₁₋₅alkyl or hydroxy-C₁₋₅alkyl;

R² is hydrogen or C₁₋₅alkyl;

R³ is hydrogen, cyano, C₁₋₅alkyl, C₁₋₅alkoxy-C₁₋₅alkyl or hydroxy-C₁₋₅alkyl;

m is an integer selected from 0, 1, 2, 3, 4 and 5;

n is 0 or 1;

with the proviso that when X is hydroxy or —CONR^{7a}R^{7b}, and n is 0, then m is an integer selected from 1, 2, 3, 4 and 5;

R⁴ is hydrogen, halogen, C₁₋₅alkyl, C₃₋₈cycloalkyl, C₃₋₈cycloalkyl-C₁₋₅alkyl, hydroxy, C₁₋₅alkoxy, C₃₋₈cycloalkyloxy or —N(R⁹)R¹⁰;

R⁵ and R⁶ are the same or different, and independently are hydrogen, halogen, C₁₋₅alkyl, C₃₋₈cycloalkyl, C₃₋₈cycloalkyl-C₁₋₅alkyl, hydroxy, C₁₋₅alkoxy, C₃₋₈cycloalkyloxy, —N(R¹¹)R¹², —CO₂R¹³, cyano, nitro, C₁₋₅alkylthio, trifluoromethyl or trifluoromethoxy; or R⁵ and R⁶ are taken together to form —CH₂—CH₂—CH₂—CH₂— or —CH═CH—CH═CH—;

with the proviso that when R⁵ and R⁶ are taken together to form —CH₂—CH₂—CH₂—CH₂—, then X is hydroxy;

R⁷ is hydrogen or C₁₋₅alkyl;

R^{7a} and R^{7b} are the same or different, and independently hydrogen or C₁₋₅alkyl;

R⁸ is hydrogen, C₁₋₅alkyl, halogen, cyano or —CO₂R¹⁴;

R⁹ and R¹⁰ are the same or different, and independently are hydrogen, C₁₋₅alkyl, C₃₋₈cycloalkyl or C₃₋₈cycloalkyl-C₁₋₅alkyl;

R¹¹ and R¹² are the same or different, and independently are hydrogen, C₁₋₅alkyl, C₃₋₈cycloalkyl or C₃₋₈cycloalkyl-C₁₋₅alkyl;

R¹³ is hydrogen or C₁₋₅alkyl;

R¹⁴ is hydrogen or C₁₋₅alkyl;

Ar is aryl or heteroaryl which aryl or heteroaryl is unsubstituted or substituted with 1 or more substituents, which are the same or different, selected from the group consisting of halogen, C₁₋₅alkyl, C₃₋₈cycloalkyl, C₂₋₅alkenyl, C₂₋₅alkynyl, C₁₋₅alkoxy, C₁₋₅alkylthio, C₁₋₅alkylsulfinyl, C₁₋₅alkylsulfonyl, cyano, nitro, hydroxy, —CO₂R¹⁵, —C(═O)R¹⁶, —CONR¹⁷R¹⁸, —OC(═O)R¹⁹, —NR²⁰CO₂R²¹, —S(O)ᵣNR²², R²³, trifluoromethyl, trifluoromethoxy, difluoromethoxy, fluoromethoxy, methylenedioxy, ethylenedioxy and —N(R²⁴)R²⁵;

R¹⁵ is hydrogen, C₁₋₅alkyl, C₃₋₈cycloalkyl or C₃₋₈cycloalkyl-C₁₋₅alkyl;

R¹⁶ is hydrogen or C₁₋₅alkyl;

R¹⁷ and R¹⁸ are the same or different, and independently are hydrogen, C₁₋₅alkyl, C₃₋₈cycloalkyl or C₃₋₈cycloalkyl-C₁₋₅alkyl;

R¹⁹ is hydrogen or C₁₋₅alkyl;

R²⁰ is hydrogen or C₁₋₅alkyl;

R²¹ is hydrogen or C₁₋₅alkyl;

R²² and R²³ are the same or different, and independently are hydrogen, C₁₋₅alkyl, C₃₋₈cycloalkyl or C₃₋₈cycloalkyl-C₁₋₅alkyl;

R²⁴ and R²⁵ are the same or different, and independently are hydrogen, C₁₋₅alkyl, C₃₋₈cycloalkyl or C₃₋₈cycloalkyl-C₁₋₅alkyl;

r is 1 or 2), individual isomers thereof, racemic or non-racemic mixtures of isomers thereof or N-oxide thereof, or pharmaceutically acceptable salts and hydrates thereof.

The terms used in the present specification have the following meanings.

The term “C₁₋₅alkyl” means a straight chain or branched chain alkyl group of 1 to 5 carbon atoms, such as methyl, ethyl, propyl, isopropyl, butyl, isobutyl, t-butyl, sec-butyl, pentyl, isopentyl or the like.

The term “C₁₋₅alkoxy” means a straight chain or branched chain alkoxy group of 1 to 5 carbon atoms, such as methoxy, ethoxy, propoxy, isopropyloxy, butoxy, isobutyloxy, pentyloxy, isopentyloxy or the like.

The term “C₁₋₅alkoxy-C₁₋₅alkyl” means a substituted C₁₋₅alkyl group having the above-mentioned C₁₋₅alkoxy group as the substituent, such as methoxymethyl, 2-methoxyethyl, 2-ethoxyethyl or the like.

The term “hydroxy-C₁₋₅alkyl” means a substituted C₁₋₅alkyl group having hydroxy group, such as hydroxymethyl, 1-hydroxyethyl, 2-hydroxyethyl, 1-hydroxypropyl, 2-hydroxypropyl, 3-hydroxypropyl, 4-hydroxybutyl, 5-hydroxypentyl or the like.

The term “C₃₋₈cycloalkyl” means a cyclic alkyl group of 3 to 8 carbon atoms, such as cyclopropyl, cyclobutyl, cyclopentyl, cyclohexyl, cycloheptyl or the like.

The term “C₃₋₈cycloalkyl-C₁₋₅alkyl” means a substituted C₁₋₅alkyl group having the above-mentioned C₃₋₈cycloalkyl as the substituent, such as cyclopropylmethyl, cyclopropylethyl, cyclopentylethyl or the like.

The term “C₃₋₈cycloalkyloxy” means a cyclic alkoxy group of 3 to 8 carbon atoms, such as cyclopropyloxy, cyclobutyloxy, cyclopentyloxy or the like.

The term “C₁₋₅alkylthio” means a straight chain or branched chain alkylthio group of 1 to 5 carbon atoms, such as methylthio, ethylthio, propylthio, isopropylthio or the like.

The term “halogen” means fluorine, chlorine, bromine or iodine atom.

The term “aryl” means a monocyclic or bicyclic group of 6 to 12 ring carbon atoms having at least one aromatic ring, such as phenyl, naphthyl or the like.

The term “heteroaryl” means a monocyclic or bicyclic group of 5 to 12 ring atoms having at least one aromatic ring having in its ring 1 to 4 atoms which may be the same or different and are selected from nitrogen, oxygen and sulfur, such as pyridyl, pyrimidinyl, imidazolyl, quinolyl, indolyl, benzofuranyl, quinoxalinyl, benzo[1,2,5]thiadiazolyl, benzo[1,2,5]oxadiazolyl or the like.

The term “C₂₋₅alkenyl” means a straight chain or branched chain alkenyl group of 2 to 5 carbon atoms, such as vinyl, isopropenyl, allyl or the like.

The term “C₂₋₅alkynyl” means a straight chain or branched chain alkynyl group of 2 to 5 carbon atoms, such as ethynyl, prop-1-ynyl, prop-2-ynyl or the like.

The term “C₁₋₅alkysulfinyl” means a straight chain or branched chain alkylsulfinyl group of 1 to 5 carbon atoms, such as methanesulfinyl, ethanesulfinyl or the like.

The term “C₁₋₅alkylsulfonyl” means a straight chain or branched chain alkylsulfonyl group of 1 to 5 carbon atoms, such as methanesulfonyl, ethanesulfonyl or the like.

The term “aryl or heteroaryl which aryl or heteroaryl is unsubstituted or substituted with 1 or more substituents, which are the same or different, selected from the group consisting of halogen, C₁₋₅alkyl, C₃₋₈cycloalkyl, C₂₋₅alkenyl, C₂₋₅alkyl, C₁₋₅alkoxy, C₁₋₅alkylthio, C₁₋₅alkylsulfinyl, C₁₋₅alkylsulfonyl, cyano, nitro, hydroxy, —CO₂R¹⁵, —C(—O)R¹⁶, —CONR¹⁷R¹⁸, —OC(═O)R¹⁹, —NR²⁰CO₂R²¹, —S(O)ᵣNR²²R²³, trifluoromethyl, trifluoromethoxy, difluoromethoxy, fluoromethoxy, methylenedioxy, ethylenedioxy and —N(R²⁴)R²⁵″ includes, for example, 2,4-dimethylphenyl, 2,6-dimethylphenyl, 2,4-dibromophenyl, 2-bromo-4-isopropylphenyl, 2,4-dichlorophenyl, 2,6-dichlorophenyl, 2-chloro-4-trifluoromethylphenyl, 4-methoxy-2-methylphenyl, 2-chloro-4-trifluoromethoxyphenyl, 4-isopropyl-2-methylthiophenyl, 2,4,6-trimethylphenyl, 4-bromo-2,6-dimethylphenyl, 4-bromo-2,6-diethylphenyl, 4-chloro-2,6-dimethylphenyl, 2,4,6-tribromophenyl, 2,4,5-tribromophenyl, 2,4,6-trichlorophenyl, 2,4,5-trichlorophenyl, 4-bromo-2,6-dichlorophenyl, 6-chloro-2,4-dibromophenyl, 2,4-dibromo-6-fluorophenyl, 2,4-dibromo-6-methylphenyl, 2,4-dibromo-6-methoxyphenyl, 2,4-dibromo-6-methylthiophenyl, 2,6-dibromo-4-isopropylphenyl, 2,6-dibromo-4-trifluoromethylphenyl, 2-bromo-4-trifluoromethylphenyl, 4-bromo-2-chlorophenyl, 2-bromo-4-chlorophenyl, 4-bromo-2-methylphenyl, 4-chloro-2-methylphenyl, 2,4-dimethoxyphenyl, 2,6-dimethyl-4-methoxyphenyl, 4-chloro-2,6-dibromophenyl, 4-bromo-2,6-difluorophenyl, 2,6-dichloro-4-trifluoromethylphenyl, 2,6-dichloro-4-trifluoromethoxyphenyl, 2,6-dibromo-4-trifluoromethoxyphenyl, 2-chloro-4,6-dimethylphenyl, 2-bromo-4,6-dimethoxyphenyl, 2-bromo-4-isopropyl-6-methoxyphenyl, 2,4-dimethoxy-6-methylphenyl, 6-dimethylamino-4-methylpyridin-3-yl, 2-chloro-6-trifluoromethylpyridin-3-yl, 2-chloro-6-trifluoromethoxypyridin-3-yl, 2-chloro-6-methoxypyridin-3-yl, 6-methoxy-2-trifluoromethylpyridin-3-yl, 2-chloro-6-difluoromethylpyridin-3-yl, 6-methoxy-2-methylpyridin-3-yl, 2,6-dimethoxypyridin-3-yl, 4,6-dimethyl-2-trifluoromethylpyrimidin-5-yl, 2-dimethylamino-6-methylpyridin-3-yl, 6-dimethylamino-2-methylpyridin-3-yl, 2,3-dihydrobenzo[1,4]dioxin-5-yl and benzo[1,3]dioxol-4-yl, 5,7-dimethylbenzo[1,2,5]thiadiazol-4-yl, 5,7-dimethylbenzo[1,2,5]oxadiazol-4-yl, 2-isopropoxy-6-trifluoromethylpyridin-3-yl, 2-methoxy-6-methylpyridin-3-yl, 2,6-dimethylpyridin-3-yl, 2-bromo-6-methoxypyridin-3-yl, 2-chloro-6-dimethylaminopyridin-3-yl, 2,6-dichloropyridin-3-yl, 2,4-dimethyl-6-dimethylaminopyridin-3-yl, 2,4,6-trimethylpyridin-3-yl, 2,4,6-trimethylpyrimidin-5-yl, 4,6-dimethyl-2-dimethylaminopyrimidin-5-yl, 5-iodo-3-methylpyridin-2-yl, 3-methyl-5-methylaminopyridin-2-yl, 3-dimethylamino-5-methylpyridin-2-yl, 5-methyl-3-methylaminopyridin-2-yl, 3-chloro-5-methylpyridin-2-yl, 3-amino-5-methylpyridin-2-yl, 5-methyl-3-nitropyridin-2-yl, 5-diethylamino-3-methylpyridin-2-yl, 5-fluoro-3-methylpyridin-2-yl, 5-chloro-3-methylpyridin-2-yl, 5-dimethylamino-3-methylpyridin-2-yl, 5-amino-3-methylpyridin-2-yl, 3-methyl-5-nitropyridin-2-yl, 3-bromo-5-methylpyridin-2-yl, 4-chloro-2,5-dimethoxyphenyl, 4,5-dimethyl-2-methoxyphenyl, 5-fluoro-2,4-dimethylphenyl, 2,4-dimethoxy-5-methylphenyl, 2-chloro-4-methoxy-5-methylphenyl, 2-chloro-5-fluoro-4-methylphenyl, 2-bromo-4,5-dimethoxyphenyl, 2-bromo-5-fluoro-4-methoxyphenyl, 2-chloro-4,5-dimethoxyphenyl, 2,5-dichloro-4-methoxyphenyl, 2,4-dichloro-5-fluorophenyl, 2-chloro-5-fluoro-4-methoxyphenyl, 2,4,5-trichlorophenyl, 2-chloro-5-fluoro-4-methylphenyl, 5-fluoro-4-methoxy-2-methylphenyl, 4,5-dimethoxy-2-methylphenyl, 5-chloro-4-methoxy-2-methylphenyl, 2,4,5-trimethylphenyl, 6-methoxy-4-methylpyridin-3-yl, 4-methoxy-6-methylpyridin-3-yl, 4,6-dimethylpyridin-3-yl, 2-chloro-4-isopropylphenyl, 2-chloro-4-methylphenyl, 4-amino-2-chlorophenyl, 2-chloro-4-dimethylcarbamoylphenyl, 2-chloro-4-methylcarbamoylphenyl, 4-carbamoyl-2-chlorophenyl, 2-chloro-4-methylsulfonylphenyl, 4-carboxy-2-chlorophenyl, 2-chloro-4-iodophenyl, 2-bromo-4-methylthiophenyl, 2-bromo-4-methylsulfinylphenyl, 2-bromo-4-dimethylaminophenyl, 2-bromo-4-methylsulfonylphenyl, 2-bromo-4-cyclopentylphenyl, 2-bromo-4-tert-butylphenyl, 2-bromo-4-propylphenyl, 2-bromo-4-methylphenyl, 2-bromo-4-trifluoromethoxyphenyl, 2-bromo-4-methoxyphenyl, 2-bromo-4-ethoxyphenyl, 4-isopropyl-2-methylsulfonylphenyl, 4-cyclopentyl-2-methylthiophenyl, 4-butyl-2-methylthiophenyl, 4-methoxy-2-methylthiophenyl, 2-methylthio-4-propylphenyl, 2-dimethylamino-4-isopropylphenyl, 2-iodo-4-isopropylphenyl, 2-fluoro-4-methylphenyl, 2,4-difluorophenyl, 2-chloro-4-methoxyphenyl, 2-chloro-4-hydroxyphenyl, 4-cyano-2-methoxyphenyl, 4-bromo-2-methoxyphenyl, 2-methoxy-4-methylphenyl, 4-chloro-2-methoxyphenyl, 2-hydroxy-4-methylphenyl, 4-fluoro-2-methoxyphenyl, 2-hydroxy-4-methylphenyl, 4-cyano-2-methoxyphenyl, 2-chloro-4-methylthiophenyl, 2-methoxy-4-trifluoromethylphenyl, 4-isopropyl-2-methoxyphenyl, 2-chloro-4-cyanophenyl, 2-chloro-4-ethoxycarbonylphenyl, 2-chloro-4-methylaminophenyl, 4-cyano-2-trifluoromethylphenyl, 4-cyano-2-methylphenyl, 2-methyl-4-trifluoromethoxyphenyl, 2-cyano-4-trifluoromethylphenyl, 4-carboxyamino-2-trifluoromethylphenyl, 4-methoxy-2-trifluoromethylphenyl, 4-fluoro-2-methylphenyl, 4-hydroxy-2-methylphenyl, 4-methoxy-2-methoxycarbonylphenyl, 2-ethyl-4-methoxyphenyl, 2-formyl-4-methoxyphenyl, 4-chloro-2-trifluoromethylphenyl, 4-dimethylamino-2-trifluoromethylphenyl, 4-difluoromethoxy-2-methylphenyl, 2-cyano-4-methoxyphenyl, 4-hydroxy-2-trifluoromethylphenyl, 4-isopropyl-2-trifluoromethylphenyl, 4-diethylamino-2-methylphenyl, 4-fluoro-2-trifluoromethylphenyl, 4-propoxy-2-trifluoromethylphenyl, 4-dimethylamino-2-methylthiophenyl, 4-isopropyl-2-isopropylthiophenyl, 2-ethylthio-4-isopropylphenyl, 4-methylamino-2-methylthiophenyl, 2-methylthio-4-propionylphenyl, 4-acetyl-2-methylthiophenyl, 4-cyano-2-methylthiophenyl, 4-methoxy-2-methylthiophenyl, 4-ethyl-2-methylthiophenyl, 4-bromo-2-methylthiophenyl, 4-isopropyl-2-methylsulfinylphenyl, 2,4-dimethylthiophenyl, 4,6-dimethyl-2-isopropylphenyl, 4,6-dimethyl-2-isopropenylphenyl, 2-acetyl-4,6-dimethylphenyl, 2,6-dimethyl-4-trifluoromethylphenyl, 2,6-dimethyl-4-isopropenylphenyl, 4-acetyl-2,6-dimethylphenyl, 2,4,6-triethylphenyl, 4,6-dimethyl-2-methylthiophenyl, 4,6-dimethyl-2-iodophenyl, 2-fluoromethoxy-4,6-dimethylphenyl, 4,6-dimethyl-2-isopropoxyphenyl, 4,6-dimethyl-2-ethoxyphenyl, 2,6-dichloro-4-ethoxyphenyl, 2-bromo-4,6-dimethoxyphenyl, 2-bromo-6-hydroxy-4-methoxyphenyl, 2,6-dibromo-4-ethoxyphenyl, 4-bromo-2-methoxy-6-methylphenyl, 2,6-dibromo-4-methoxyphenyl, 4,6-dibromo-2-trifluoromethoxyphenyl, 2,4-dibromo-6-trifluoromethylphenyl, 4-bromo-2-chloro-6-methylphenyl, 4-chloro-2,6-dimethoxyphenyl, 2,4-dichloro-6-methoxyphenyl, 4,6-dichloro-2-methylthiophenyl, 4,6-dichloro-2-trifluoromethylphenyl, 2,6-dimethoxy-4-ethylphenyl, 4,6-dimethyl-2-methoxyphenyl, 2,6-dimethoxy-4-methylphenyl, 2-chloro-6-methoxy-4-methylphenyl, 4,6-dimethyl-2-ethoxyphenyl, 6-hydroxy-2,4-dimethylphenyl, 4-cyano-2-methoxy-6-methylphenyl, 6-fluoro-2-methoxy-4-methylphenyl, 4-acetyl-2-methoxy-6-methylphenyl, 2-chloro-4,6-dimethoxyphenyl, 2,6-dimethoxy-4-ethoxyphenyl, 2,4,6-trimethoxyphenyl, 4,6-dibromo-2-trifluoromethoxyphenyl, 2-bromo-4-dimethylamino-6-methoxyphenyl, 4-bromo-2-methoxy-6-methylphenyl, 4,6-dimethoxy-2-propoxyphenyl, 4,6-dichloro-2-propoxyphenyl, 2-bromo-6-hydroxy-4-methoxyphenyl, 2,4,6-trifluorophenyl, 2-bromo-6-fluoro-4-methylphenyl, 4-difluoromethoxy-2,6-dimethylphenyl, 2,6-dimethyl-4-ethoxyphenyl, 2,6-dimethyl-4-isopropoxyphenyl, 2,6-dimethyl-4-methylthiophenyl, 2,6-dimethyl-4-methylsulfonylophenyl, 2,6-dimethyl-4-methylsulfinylophenyl, 2,3-dichlorophenyl, 4-methoxy-2,3-dimethylphenyl, 2-chloro-3-fluoro-4-methoxyphenyl, 2,3,4-trichlorophenyl, 4-methoxy-2,5-dimethylphenyl.

The “pharmaceutically acceptable salts” in the present invention include, for example, salts with an inorganic acid such as sulfuric acid, hydrochloric acid, hydrobromic acid, phosphoric acid, nitric acid or the like; salts with an organic acid such as acetic acid, oxalic acid, lactic acid, tartaric acid, fumaric acid, maleic acid, citric acid, benzenesulfonic acid, methanesulfonic acid, p-toluenesulfonic acid, benzoic acid, camphorsulfonic acid, ethanesulfonic acid, glucoheptonic acid, gluconic acid, glutamic acid, glycolic acid, malic acid, malonic acid, mandelic acid, galactaric acid, naphthalene-2-sulfonic acid or the like; salts with one or more metal ions such as lithium ion, sodium ion, potassium ion, calcium ion, magnesium ion, zinc ion, aluminium ion or the like; salts with amines such as ammonia, arginine, lysine, piperazine, choline, diethylamine, 4-phenylcyclohexylamine, 2-aminoethanol, benzathine or the like.

A compound of the present invention includes any isomers such as diastereomers, enantiomers, geometric isomers and tautomeric forms. In a compound represented by formula [I], if the cyclic amino group has one or more chiral carbons and/or if there is an axial chirality between Ar and pyrrolopyrimidine (or pyrrolopyridine) ring, several stereoisomers (diastereomers or enantiomers) can exist. The compound of the present invention includes all of the individual isomers and the racemic and non-racemic mixtures of the isomers.

Preferable examples of the compound of the present invention are as follows.

That is, preferable are compounds represented by the following formula [I]: 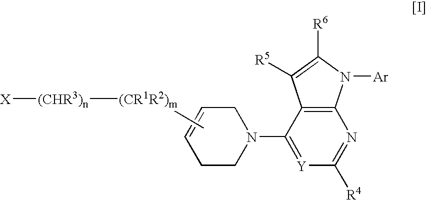
(wherein the tetrahydropyridine is represented by the following formula [II]:

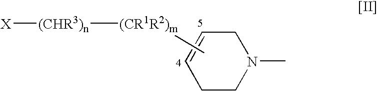

in which the tetrahydropyridine ring is substituted with a group represented by —(CR¹R²)ₘ—(CHR³)ₙ—X at the 4-position or 5-position of the tetrahydropyridine ring;

X is hydroxy, cyano or —CO₂R⁷;

Y is N or CR⁸;

with the proviso that when Y is CR⁸, then X is hydroxy;

R¹ is hydrogen, hydroxy, C₁₋₅alkyl, C₁₋₅alkoxy-C₁₋₅alkyl or hydroxy-C₁₋₅alkyl;

R² is hydrogen or C₁₋₅alkyl;

R³ is hydrogen, cyano, C₁₋₅alkyl, C₁₋₅alkoxy-C₁₋₅alkyl or hydroxy-C₁₋₅alkyl;

m is an integer selected from 0, 1, 2, 3, 4 and 5;

n is 0 or 1;

with the proviso that when X is hydroxy, and n is 0, then m is an integer selected from 1, 2, 3, 4 and 5;

R⁴ is hydrogen, C₁₋₅alkyl, C₃₋₈cycloalkyl, C₃₋₈-cycloalkyl-C₁₋₅alkyl, hydroxy, C₁₋₅alkoxy, C₃₋₈cycloalkyloxy or —N(R⁹)R¹⁰;

R⁵ and R⁶ are the same or different, and independently are hydrogen, halogen, C₁₋₅alkyl, C₃₋₈cycloalkyl, C₃₋₈cycloalkyl-C₁₋₅alkyl, hydroxy, C₁₋₅alkoxy, C₃₋₈cycloalkyloxy, —N(R¹¹)R¹², —CO₂R¹³, cyano, nitro, C₁₋₅alkylthio, trifluoromethyl or trifluoromethoxy; or R⁵ and R⁶ are taken together to form —CH₂—CH₂—CH₂—CH₂— or —CH═CH—CH═CH—;

with the proviso that when R⁵ and R⁶ are taken together to form —CH₂—CH₂—CH₂—CH₂—, then X is hydroxy;

R⁷ is hydrogen or C₁₋₅alkyl;

R⁸ is hydrogen, C₁₋₅alkyl, halogen, cyano or —CO₂R¹⁴;

R⁹ and R¹⁰ are the same or different, and independently are hydrogen, C₁₋₅alkyl, C₃₋₈cycloalkyl or C₃₋₈cycloalkyl-C₁₋₅alkyl;

R¹¹ and R¹² are the same or different, and independently are hydrogen, C₁₋₅alkyl, C₃₋₈cycloalkyl or C₃₋₈cycloalkyl-C₁₋₅alkyl;

R¹³ is hydrogen or C₁₋₅alkyl;

R¹⁴ is hydrogen or C₁₋₅alkyl;

Ar is aryl or heteroaryl which aryl or heteroaryl is unsubstituted or substituted with 1 or more substituents, which are the same or different, selected from the group consisting of halogen, C₁₋₅alkyl, C₃₋₈cycloalkyl, C₂₋₅alkenyl, C₂₋₅alkynyl, C₁₋₅alkoxy, C₁₋₅alkylthio, C₁₋₅alkylsulfinyl, C₁₋₅alkylsulfonyl, cyano, nitro, hydroxy, —CO₂R¹⁵, —C(═O)R¹⁶, —CONR¹⁷R¹⁸, —OC(═O)R¹⁹, —NR²⁰CO₂R²¹, —S(O)ᵣNR²²R²³, trifluoromethyl, trifluoromethoxy, difluoromethoxy, fluoromethoxy, methylenedioxy, ethylenedioxy and —N(R²⁴)R²⁵;

R¹⁵ is hydrogen, C₁₋₅alkyl, C₃₋₈cycloalkyl or C₃₋₈cycloalkyl-C₁₋₅alkyl;

R¹⁶ is hydrogen or C₁₋₅alkyl;

R¹⁷ and R¹⁸ are the same or different, and independently are hydrogen, C₁₋₅alkyl, C₃₋₈cycloalkyl or C₃₋₈cycloalkyl-C₁₋₅alkyl;

R¹⁹ is hydrogen or C₁₋₅alkyl;

R²⁰ is hydrogen or C₁₋₅alkyl;

R²¹ is hydrogen or C₁₋₅alkyl;

R²² and R²³ are the same or different, and independently are hydrogen, C₁₋₅alkyl, C₃₋₈cycloalkyl or C₃₋₈cycloalkyl-C₁₋₅alkyl;

R²⁴ and R²⁵ are the same or different, and independently are hydrogen, C₁₋₅alkyl, C₃₋₈cycloalkyl or C₃₋₈cycloalkyl-C₁₋₅alkyl;

r is 1 or 2), individual isomers thereof, racemic or non-racemic mixtures of isomers thereof or N-oxide thereof, or pharmaceutically acceptable salts and hydrates thereof.

More preferable are compounds represented by the formula [I] in which Y is N. More preferable are compounds represented by the formula [I] in which Y is N; X is hydroxy; m is an integer selected from 1, 2, 3, 4 and 5; n is 0; R¹ and R² are hydrogen. More preferable are compounds represented by the formula [I] in which Y is N; X is hydroxy; m is an integer selected from 1, 2 and 3; n is 0; R¹ and R² are hydrogen; R⁴ is C₁₋₅alkyl; R⁵ and R⁶ are the same or different, and independently are hydrogen or C₁₋₅alkyl; Ar is phenyl which phenyl is substituted with two or three substituents, which are the same or different, selected from the group consisting of halogen, C₁₋₃alkyl, C₁₋₃alkoxy, C₁₋₃alkylthio, trifluoromethyl, trifluoromethoxy and —N(R²⁴)R (wherein R²⁴ and R²⁵ are the same or different, and independently are hydrogen or C₁₋₃alkyl).

Other preferable are compounds represented by the formula [I] in which Y is N; X is cyano. More preferable are compounds represented by the formula [I] in which Y is N; X is cyano; m is 0 or 1; n is 0; R¹ and R² are hydrogen; R⁴ is C₁₋₅alkyl; R⁵ and R⁶ are the same or different, and independently are hydrogen or C₁₋₅alkyl; Ar is phenyl which phenyl is substituted with two or three substituents, which are the same or different, selected from the group consisting of halogen, C₁₋₃alkyl, C₁₋₃alkoxy, C₁₋₃alkylthio, trifluoromethyl, trifluoromethoxy and —N(R²⁴)R²⁵ (wherein R²⁴ and R²⁵ are the same or different, and independently are hydrogen or C₁₋₃alkyl).

Other preferable are compounds represented by the formula [I] in which Y is CR⁸; X is hydroxy. More preferable are compounds represented by the formula [I] in which Y is CH; X is hydroxy; m is an integer selected from 1, 2, 3, 4 and 5; n is 0; R¹ and R² are hydrogen. More preferable are compounds represented by the formula [I] in which Y is CH; X is hydroxy; m is an integer selected from 1, 2 and 3; n is 0; R¹ and R² are hydrogen; R⁴ is C₁₋₅alkyl; R⁵ and R⁶ are the same or different, and independently are hydrogen or C₁₋₅alkyl; Ar is phenyl which phenyl is substituted with two or three substituents, which are the same or different, selected from the group consisting of halogen, C₁₋₃alkyl, C₁₋₃alkoxy, C₁₋₃alkylthio, trifluoromethyl, trifluoromethoxy and —N(R²⁴)R²⁵ (wherein R²⁴ and R²⁵ are the same or different, and independently are hydrogen or C₁₋₃alkyl.

The preferable R¹ is hydrogen.

The preferable R² is hydrogen.

The preferable R³ is hydrogen.

The preferable R⁴ is C₁₋₃ alkyl. The more preferable R⁴ is methyl.

The preferable R⁵ is C₁₋₃ alkyl. The more preferable R⁵ is methyl.

The preferable R⁶ is hydrogen or C₁₋₃ alkyl. The more preferable R⁶ is hydrogen or methyl.

When X is hydroxy, preferable m is an integer selected from 1, 2 and 3 and preferable n is 0.

When X is cyano, preferable m is 0 or 1 and preferable n is 0.

The preferable Ar is phenyl which phenyl is substituted with two or three substituents, which are the same or different, selected from the group consisting of chloro, bromo, C₁₋₃alkyl, C₁₋₃alkoxy, C₁₋₃alkylthio, trifluoromethyl, trifluoromethoxy and dimethylamino. The more preferable Ar is phenyl which phenyl is substituted with two or three substituents, which are the same or different, selected from the group consisting of chloro, bromo, C₁₋₃alkyl.

The compound represented by the formula [I] can be produced, for example, by the process shown in the following reaction schemes 1 and 2 [in the following reaction schemes, R¹, R², R³, R⁴, R⁵, R⁶, m, n, X, Y and Ar are as defined above; L¹ is chloro, bromo, iodo, methanesulfonyloxy, benzenesulfonyloxy, p-toluenesulfonyloxy or trifluoromethanesulfonyloxy group; X^{a} is hydroxy, cyano, —C(═O)O—C₁₋₅alkyl or —CONR^{7a}R^{7b}; R^{a} is C₁₋₅alkyl; R^{b} is C₁₋₅alkyl or phenyl; R^{c} is C₁₋₅alkoxy or —NR^{7a}R^{7b}.].
Reaction Scheme 1 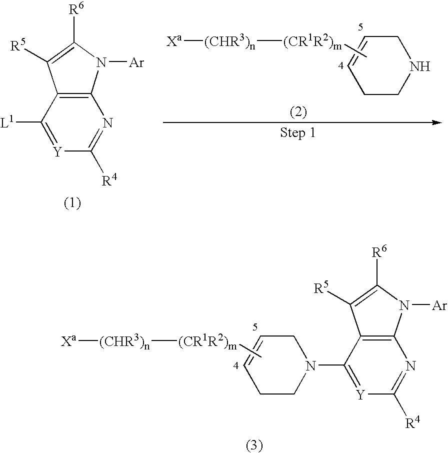
Step 1:

Compound (3), a compound of the present invention, can be obtained by reacting Compound (1) with Compound (2) in an inert solvent or no solvent in the presence or absence of a base. Herein, the base includes, for example, amines such as triethylamine, N,N-diisopropylethylamine, pyridine and the like; inorganic bases such as sodium carbonate, potassium carbonate, sodium hydrogencarbonate, potassium hydrogencarbonate, sodium hydroxide, potassium hydroxide, barium hydroxide, sodium hydride and the like; metal alcoholates such as sodium methoxide, sodium ethoxide, potassium tert-butoxide and the like; metal amides such as sodium amide, lithium diisopropylamide and the like; and Grignard reagents such as methylmagnesiumn bromide and the like. The inert solvent includes, for example, alcohols such as methanol, ethanol, isopropyl alcohol, ethylene glycol and the like; ethers such as diethyl ether, tetrahydrofuran, 1,4-dioxane, 1,2-dimethoxyethane and the like; hydrocarbons such as benzene, toluene, xylene and the like; esters such as ethyl acetate, ethyl formate and the like; amides such as N,N-dimethylformamide, N-methylpyrrolidone, N,N-dimethylacetamide and the like; acetonitrile; dimethyl sulfoxide; pyridine; chloroform; dichloromethane; water; and mixtures of solvents selected from these inert solvents.
Reaction Scheme 2

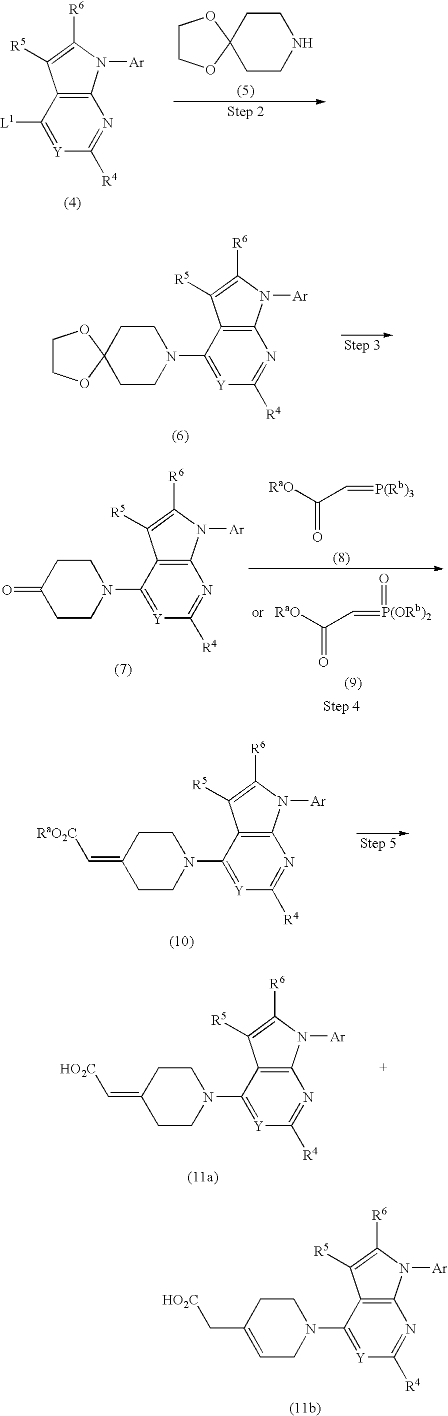

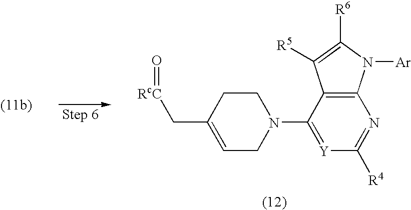

Compound (6) can be obtained by reacting Compound (4) with Compound (5) in an inert solvent or without any solvent in the presence or absence of a base. Herein, the base includes, for example, amines such as triethylamine, N,N-diisopropylethylamine, pyridine and the like; inorganic bases such as sodium carbonate, potassium carbonate, sodium hydrogencarbonate, potassium hydrogencarbonate, sodium hydroxide, potassium hydroxide, barium hydroxide, sodium hydride and the like; metal alcoholates such as sodium methoxide, sodium ethoxide, potassium tert-butoxide and the like; metal amides such as sodium amide, lithium diisopropylamide and the like; and Grignard reagents such as methylmagnesium bromide and the like. The inert solvent includes, for example, alcohols such as methanol, ethanol, isopropyl alcohol, ethylene glycol and the like; ethers such as diethyl ether, tetrahydrofuran, 1,4-dioxane, 1,2-dimethoxyethane and the like; hydrocarbons such as benzene, toluene, xylene and the like; esters such as ethyl acetate, ethyl formate and the like; amides such as N,N-dimethylformamide, N-methylpyrrolidone, N,N-dimethylacetamide and the like; acetonitrile; dimethyl sulfoxide; pyridine; chloroform; dichloromethane; water; and mixtures of solvents selected from these inert solvents.

Step 3:

Compound (6) can be converted to Compound (7) by converting the acetal to the ketone by using a method as described in Protective Group in Organic Synthesis (T. W. Greene, P. G. M. Wuts; 3^{rd} ed., 1999, John Wiley & sons, Inc.).

Step 4:

Compound (7) can be converted to Compound (10) by reacting Compound (7) with Compound (8) or Compound (9) in an inert solvent in the presence or absence of a base. Herein, the base includes, for example, amines such as triethylamine, N,N-diisopropylethylamine, pyridine and the like; inorganic bases such as sodium carbonate, potassium carbonate, sodium hydrogencarbonate, potassium hydrogencarbonate, sodium hydroxide, potassium hydroxide, barium hydroxide, sodium hydride and the like; metal alcoholates such as sodium methoxide, sodium ethoxide, potassium tert-butoxide and the like; metal amides such as sodium amide, lithium diisopropylamide and the like; and Grignard reagents such as methylmagnesium bromide and the like. The inert solvent includes, for example, alcohols such as methanol, ethanol, isopropyl alcohol, ethylene glycol and the like; ethers such as diethyl ether, tetrahydrofuran, 1,4-dioxane, 1,2-dimethoxyethane and the like; hydrocarbons such as benzene, toluene, xylene and the like; esters such as ethyl acetate, ethyl formate and the like; amides such as N,N-dimethylformamide, N-methylpyrrolidone, N,N-dimethylacetamide and the like; acetonitrile; dimethyl sulfoxide; pyridine; chloroform; dichloromethane; water; and mixtures of solvents selected from these inert solvents.

Step 5:

A mixture of Compound (11a) and Compound (11b) can be obtained by conventional hydrolysis method of the ester from Compound (10) with an acid or a base in an inert solvent. Herein, the acid includes, for example, inorganic acids such as sulfuric acid, hydrochloric acid, hydrobromic acid, nitric acid or the like; organic acids such as formic acid, acetic acid, trifluoroacetic acid, benzenesulfonic acid, methanesulfonic acid, p-toluenesulfonic acid, trifluoromethanesulfonic acid and the like. The base includes, for example, inorganic bases such as sodium carbonate, potassium carbonate, sodium hydrogencarbonate, potassium hydrogencarbonate, sodium hydroxide, potassium hydroxide, barium hydroxide and the like; The inert solvent includes, for example, alcohols such as methanol, ethanol, isopropyl alcohol, ethylene glycol and the like; ethers such as diethyl ether, tetrahydrofuran, 1,4-dioxane, 1,2-dimethoxyethane and the like; hydrocarbons such as benzene, toluene, xylene and the like; esters such as ethyl acetate, ethyl formate and the like; amides such as N,N-dimethylformamide, N-methylpyrrolidone, N,N-dimethylacetamide and the like; acetonitrile; dimethyl sulfoxide; pyridine; chloroform; dichloromethane; water; and mixtures of solvents selected from these inert solvents.

Step 6:

Compound (12), a compound of the present invention, can be synthesized from Compound (11b) by conventional methods for amidating a carboxy group, esterification of a carboxy group or alkylation of a carboxy group in the presence or absence of a base in an inert solvent. Conventional methods for amidating a carboxy group or esterification of a carboxy group are: for example, the reaction aria a mixed acid anhydride obtained by the reaction of Compound (11b) with haloformic acid ester (e.g., ethyl chloroformate or isobutyl chloroformate) or an acid chloride (e.g., benzoyl chloride or pivaloyl chloride); the reaction in the presence of a condensing agent such as N,N′-dicyclohexylcarbodiimide (DCC), 1-(3-dimethylaminopropyl)-3-ethylcarbodiimide hydrochloride (EDCl), carbonyldiimidazole (CDI), diphenylphosphorylazide (DPPA), diethyl cyanophosphate or the like, and optionally an additive such as 1-hydroxybenzotriazole (HOBt), N-hydroxysuccinimide, 4-dimethylaminopyridine or the like; or the reaction via an acid halide obtained by the reaction of Compound (11b) with a halogenating reagent such as thionyl chloride, oxalyl chloride, or the like; conventional methods for alkylation of a carboxy group is the reaction with an alkylating reagent such as alkylhalide or alkylsulfonate in the presence or absence of an additive to accelerate the reaction such as NaI and KI. The base includes amines such as triethylamine, N,N-diisopropylethylamine, pyridine, 1,8-diazabicyclo[5.4.0]undec-7-ene and the like; inorganic bases such as sodium carbonate, potassium carbonate, sodium hydrogencarbonate, potassium hydrogencarbonate, sodium hydroxide, potassium hydroxide, lithium hydroxide, barium hydroxide, sodium hydride and the like. The inert solvent includes, for example, alcohols such as methanol, ethanol, isopropyl alcohol, ethylene glycol and the like; ethers such as diethyl ether, tetrahydrofuran, 1,4-dioxane, 1,2-dimethoxyethane and the like; hydrocarbons such as benzene, toluene and the like; amides such as N,N-dimethylformamide, N-methylpyrrolidone, N,N-dimethylacetamide and the like; acetonitrile; dimethyl sulfoxide; pyridine; chloroform; dichloromethane; water; and mixtures of solvents selected from these inert solvents.

The compound of the present invention can be converted to a salt in an inert solvent with an inorganic acid such as sulfuric acid, hydrochloric acid, hydrobromic acid, phosphoric acid, nitric acid or the like, with an organic acid such as acetic acid, oxalic acid, lactic acid, tartaric acid, fumaric acid, maleic acid, citric acid, benzenesulfonic acid, methanesulfonic acid, p-toluenesulfonic acid, benzoic acid, camphorsulfonic acid, ethanesulfonic acid, glucoheptonic acid, gluconic acid, glutamic acid, glycolic acid, malic acid, malonic acid, mandelic acid, galactaric acid, naphthalene-2-sulfonic acid or the like, with an inorganic base such as lithium hydroxide, sodium hydroxide, potassium hydroxide, calcium hydroxide, magnesium hydroxide, zinc hydroxide, aluminum hydroxide or the like or with an organic base such as ammonia, arginine, lysine, piperazine, choline, diethylamine, 4-phenylcyclohexylamine, 2-aminoethanol, benzathine or the like. The inert solvent includes, for example, alcohols such as methanol, ethanol, isopropyl alcohol, ethylene glycol and the like; ethers such as diethyl ether, tetrahydrofuran, 1,4-dioxane, 1,2-dimethoxyethane and the like; hydrocarbons such as benzene, toluene and the like; esters such as ethyl acetate, ethyl formate and the like; ketones such as acetone, methylethylketone and the like; amides such as N,N-dimethylformamide, N-methylpyrrolidone, N,N-dimethylacetamide and the like; acetonitrile; dichloromethane; chloroform; dimethyl sulfoxide; pyridine; water; and mixtures of solvents selected from these inert solvents.

The compound of the present invention is useful as a therapeutic or prophylactic agent for diseases in which CRF is considered to be involved. For this purpose, the compound of the present invention can be formulated into tablets, pills, capsules, granules, powders, solutions, emulsions, suspensions, injections and the like by a conventional preparation technique by adding conventional fillers, binders, disintegrators, pH-adjusting agents, solvents, etc.

The compound of the present invention can be administered to an adult patient in a dose of 0.1 to 500 mg per day in one portion or several portions orally or parenterally. The dose can be properly increased or decreased depending on the kind of a disease and the age, body weight and symptom of a patient.

### EMBODIMENTS OF THE INVENTION

The present invention is concretely explained with reference to the following examples and test example, but is not limited thereto.

### Example 1

### Synthesis of 2-{1-[1-(4-bromo-2,6-dimethylphenyl)-3,6-dimethyl-1H-pyrrolo[2,3-b]pyridin-4-yl]-1,2,3,6-tetrahydropyridin-4-yl}ethanol (compound 1-014)

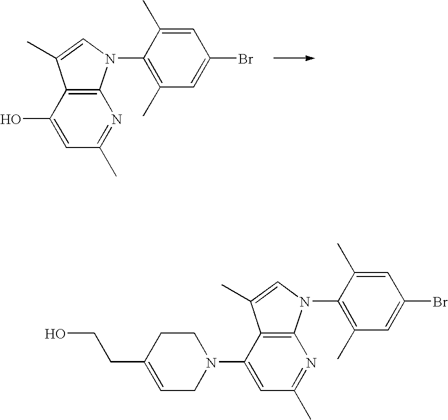

A suspension of 1-(4-bromo-2,6-dimethylphenyl)-3,6-dimethyl-1H-pyrrolo[2,3-b]pyridin-4-ol (1.0 g), triethylamine (0.61 g) in CHCl₃ (20 mL), trifluoromethanesulfonic anhydride (0.61 mL) was added with cooling in an ice bath and the mixture was stirred for 30 minutes. A saturated aqueous NaHCO₃ solution was added to the reaction mixture and separated. The organic layer was washed with brine, dried over Na₂SO₄ and filtered. The filtrate was concentrated under reduced pressure to obtain crude trifluoromethanesulfonic acid 1-(4-bromo-2,6-dimethylphenyl)-3,6-dimethyl-1H-pyrrolo[2,3-b]pyridin-4-yl ester (2.19 g). The crude trifluoromethanesulfonic acid 1-(4-bromo-2,6-dimethylphenyl)-3,6-dimethyl-1H-pyrrolo[2,3-b]pyridin-4-yl ester was dissolved in N-methylpyrrolidone (1.5 mL) and then 2-(1,2,3,6-tetrahydropyridin-4-yl)-ethanol (2.5 mL) and N,N-diisopropylethylamine (2.3 g) were added. The mixture was heated at 140° C. for 4 hours in a sealed tube. After cooling to room temperature, the reaction mixture was poured into a mixture of ethyl acetate and a saturated aqueous NaHCO₃ solution, and separated. The organic layer was washed with brine, dried over Na₂SO₄ and filtered. The filtrate was concentrated under reduced pressure and the residue was purified with column chromatography (silica gel eluent: hexane:ethyl acetate=1/1) to obtain a solid. The solid was washed with ethyl acetate to give the title compound (25 mg).

### Example 2

### Synthesis of 2-{1-[7-(4-bromo-2,6-dimethylphenyl)-2,5-dimethyl-7H-pyrrolo[2,3-d]pyrimidin-4-yl]-1,2,3,6-tetrahydropyridin-4-yl}ethanol (compound 1-013)

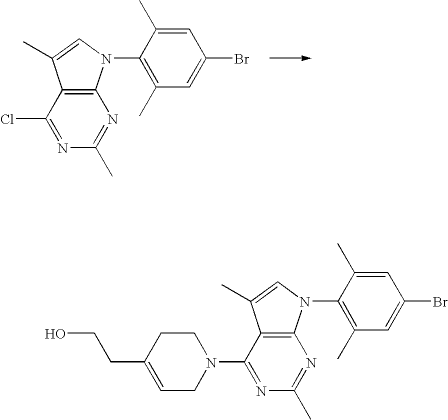

A mixture of 7-(4-bromo-2,6-dimethylphenyl)-4-chloro-2,5-dimethyl-7H-pyrrolo[2,3-d]pyrimidine (1.0 g), 2-(1,2,3,6-tetrahydropyridin-4-yl)-ethanol (0.9 g) and N,N-diisopropylethylamine (1.1 g) was heated at 100° C. for 5 hours in a sealed tube. After cooling to room temperature, the reaction mixture was poured into a mixture of ethyl acetate and a saturated aqueous NaHCO₃ solution, and separated. The organic layer was washed with brine, dried over Na₂SO₄ and filtered. The filtrate was concentrated under reduced pressure and the residue was purified with column chromatography (silica gel eluent: hexane:ethyl acetate=2/1) to obtain an solid. The solid was washed with ethyl acetate to give the title compound (69 mg).

### Example 3

### Synthesis of {1-[7-(2,6-dibromo-4-trifluoromethyl-phenyl)-2,5,6-trimethyl-7H-pyrrolo[2,3-d]pyrimidin-4-yl]-1,2,3,6-tetrahydro-pyridin-4-yl}-acetic acid (compound 1-015)

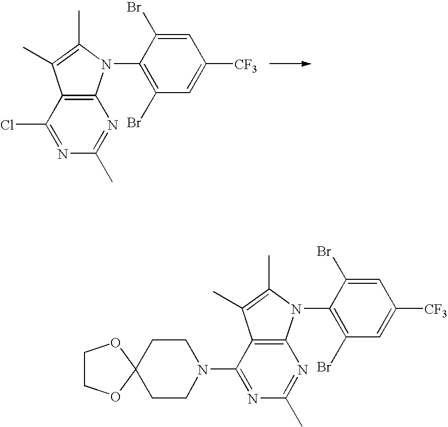 (1) A mixture of 4-chloro-7-(2,6-dibromo-4-trifluoromethyl-phenyl)-2,5,6-trimethyl-7H-pyrrolo[2,3-d]pyrimidine (5.0 g) and 4-piperidone ethylene ketal (3.0 g) in ethylene glycol (25 ml) was heated at 150° C. for 30 minutes. After cooling to room temperature, the reaction mixture was poured into a mixture of ethyl acetate and a saturated aqueous NaHCO₃ solution, and separated. The organic layer was washed with water three times and brine, dried over Na₂SO₄ and filtered. The filtrate was concentrated under reduced pressure to give a solid and the solid was washed with isopropyl ether to give 8-[7-(2,6-dibromo-4-trifluoromethyl-phenyl)-2,5,6-trimethyl-7H-pyrrolo[2,3-d]pyrimidin-4-yl]-1,4-dioxa-8-aza-spiro[4.5]decane (3.87 g). 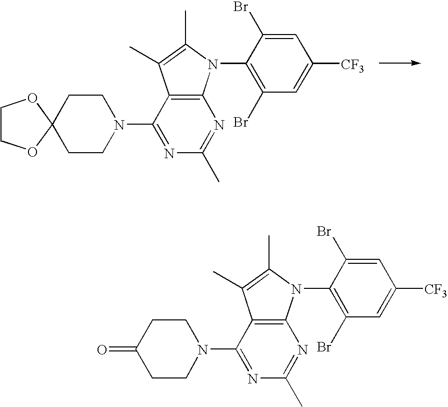
(2) A mixture of 8-[7-(2,6-dibromo-4-trifluoromethyl-phenyl)-2,5,6-trimethyl-7H-pyrrolo[2,3-d]pyrimidin-4-yl]-1,4-dioxa-8-aza-spiro[4.5]decane (3.77 g) and 2.9 M HCl (10 ml) in THF (10 ml) was stirred at room temperature for 17 hours. To the mixture was added 2.9 M HCl (10 ml) and heated at 40° C. for 5 hours. The solvent was distilled off under reduced pressure, and the residue was made basic with a saturated aqueous NaHCO₃ solution, and extracted with ethyl acetate three times. The organic layer was dried over Na₂SO₄ and filtered. The filtrate was concentrated under reduced pressure and the residue was purified with column chromatography (silica gel: Wako gel C200, eluent: hexane:ethyl acetate=9/1) to obtain 1-[7-(2,6-dibromo-4-trifluoromethyl-phenyl)-2,5,6-trimethyl-7H-pyrrolo[2,3-d]pyrimidin-4-yl]-piperidin-4-one (3.7 g) as amorphous. 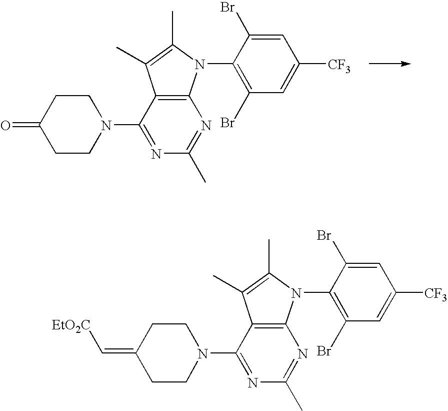
(3) To a suspension of 60% NaH (273 mg) in THF (10 ml) was added ethyl diethyl phosphonoacetate (1.7 g) under ice-cooling over a period of 3 minutes. The ice bath was removed, and the mixture was stirred at room temperature for 15 minutes. To the mixture was added a solution of 1-[7-(2,6-dibromo-4-trifluoromethyl-phenyl)-2,5,6-trimethyl-7H-pyrrolo[2,3-d]pyrimidin-4-yl]-piperidin-4-one (3.49 g) in THF (10 ml) at room temperature over a period of 5 minutes and the mixture was stirred for 30 min. To the mixture was added a saturated aqueous NH₄Cl solution, and the THF was distilled off under reduced pressure. The residue was partitioned between ethyl acetate and brine, and the organic layer was dried over Na₂SO₄ and filtered. The filtrate was concentrated under reduced pressure and the residue was purified with column chromatography (silica gel: Wako gel C200, eluent: hexane:ethyl acetate=5/1) to obtain {1-[7-(2,6-dibromo-4-trifluoromethyl-phenyl)-2,5,6-trimethyl-7H-pyrrolo[2,3-d]pyrimidin-4-yl]-piperidin-4-ylidene}-acetic acid ethyl ester (3.83 g) as amorphous. 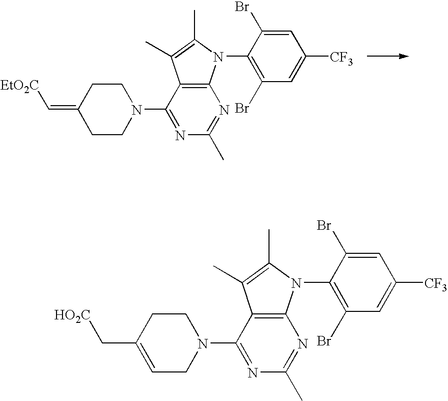
(4) A mixture of {1-[7-(2,6-dibromo-4-trifluoromethyl-phenyl)-2,5,6-trimethyl-7H-pyrrolo[2,3-d]pyrimidin-4-yl]-piperidin-4-ylidene}-acetic acid ethyl ester (2.22 g) and KOH (929 mg) in a mixture of water (1 ml) and EtOH (8 ml) was heated at 80° C. for 1 hour. The reaction mixture was neutralized with 10% HCl under ice-cooling and the solid precipitated was collected by filtration to obtain a mixture of {1-[7-(2,6-dibromo-4-trifluoromethyl-phenyl)-2,5,6-trimethyl-7H-pyrrolo[2,3-d]pyrimidin-4-yl]-piperidin-4-ylidene}-acetic acid and {1-[7-(2,6-dibromo-4-trifluoromethyl-phenyl)-2,5,6-trimethyl-7H-pyrrolo[2,3-d]pyrimidin-4-yl]-1,2,3,6-tetrahydro-pyridin-4-yl}-acetic acid. The mixture was separated and purified with column chromatography (silica gel: Wako gel C200, eluent: CHCl₃:MeOH=40/1) to obtain the title compound (0.40 g) as a solid.

### Example 4

### Synthesis of 2-{1-[7-(2,6-dibromo-4-trifluoromethyl-phenyl)-2,5,6-trimethyl-7H-pyrrolo[2,3-d]pyrimidin-4-yl]-1,2,3,6-tetrahydro-pyridin-4-yl}-N-methyl-acetamide (compound 1-017)

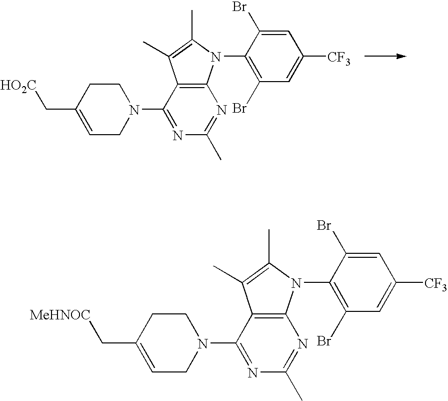

To a solution of {1-[7-(2,6-dibromo-4-trifluoromethyl-phenyl)-2,5,6-trimethyl-7H-pyrrolo[2,3-d]pyrimidin-4-yl]-1,2,3,6-tetrahydro-pyridin-4-yl}-acetic acid (175 mg), 1-(3-dimethylaminopropyl)-3-ethylcarbodiimide hydrochloride (67 mg) and 1-hydroxybenzotriazole (67 mg) in DMF (1 ml) was added 40% methylamine in water (30 ul) at room temperature and the mixture was stirred at room temperature for 12 hours. The reaction mixture was diluted with ethyl acetate, and washed with a saturated aqueous NH₄Cl solution, water and a saturated aqueous NaHCO₃ solution, dried over Na₂SO₄ and filtered. The filtrate was concentrated under reduced pressure and the residue was purified with column chromatography (silica gel: Wako gel C200, eluent: CHCl₃:MeOH 30/1) to obtain a solid. The solid was washed with isopropyl ether to give the title compound (89 mg).

TABLE 1*¹

| | |
|---|---|
| | [I] |
| 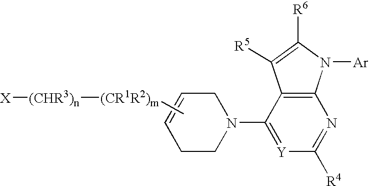 | |

| Com. No. | Ex. No | 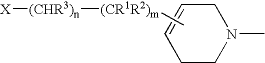 | Y | R⁴ | R⁵ | R⁶ | —Ar | melting point (° C.) (solvent for crystallization) |
|---|---|---|---|---|---|---|---|---|
| 1-001 | 2 | 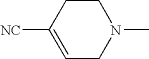 | N | CH₃ | CH₃ | CH₃ | 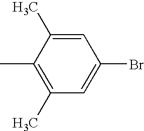 | 218-219*² |
| 1-002 | 2 | 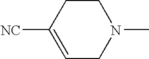 | N | CH₃ | CH₃ | H | 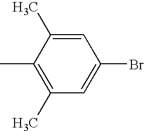 | 179-181 (EtOAc)*³ |
| 1-003 | 2 | 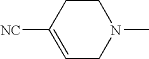 | N | CH₃ | CH₃ | CH₃ | 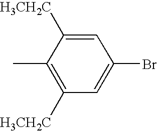 | 135-137 (IPE/hexane) |
| 1-004 | 2 | 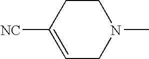 | N | CH₃ | CH₃ | H | 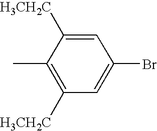 | 177-179 (IPE) |
| 1-005 | 2 | 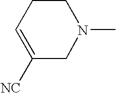 | N | CH₃ | CH₃ | CH₃ | 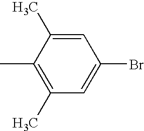 | 170-172 (IPE) |
| 1-006 | 2 | 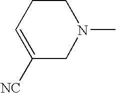 | N | CH₃ | CH₃ | H | 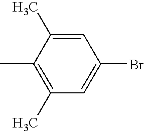 | 209-211 (IPE) |
| 1-007 | 2 | 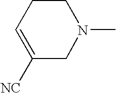 | N | CH₃ | CH₃ | CH₃ | 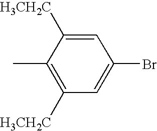 | amorphous |
| 1-008 | 2 | 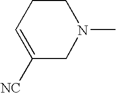 | N | CH₃ | CH₃ | H | 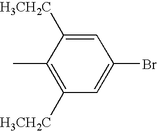 | amorphous |
| 1-009 | 2 | 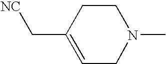 | N | CH₃ | CH₃ | CH₃ | 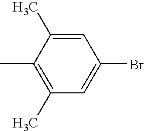 | 137-139 (IPE) |
| 1-010 | 2 | 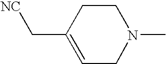 | N | CH₃ | CH₃ | H | 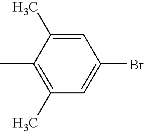 | 155-156 (IPE) |
| 1-011 | 2 | 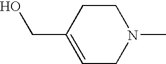 | N | CH₃ | CH₃ | H | 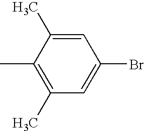 | 209-210 |
| 1-012 | 1 | 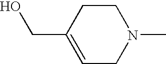 | CH | CH₃ | CH₃ | H | 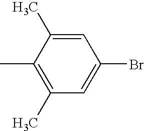 | 159-161 (IPE/EtOAc) |
| 1-013 | 2 | 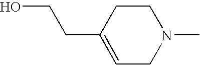 | N | CH₃ | CH₃ | H | 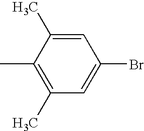 | 172-174 (EtOAc) |
| 1-014 | 1 | 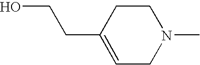 | CH | CH₃ | CH₃ | H | 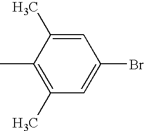 | 181-183 (EtOAc) |
| 1-015 | 3 | 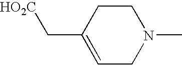 | N | CH₃ | CH₃ | CH₃ | 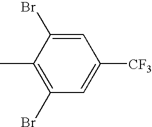 | amorphous |
| 1-016 | 3 | 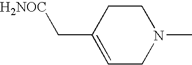 | N | CH₃ | CH₃ | CH₃ | 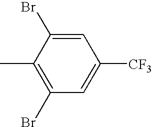 | 156-157 (IPE) |
| 1-017 | 3 | 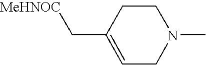 | N | CH₃ | CH₃ | CH₃ | 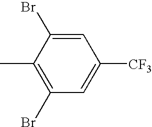 | 180-183 (IPE) |
| 1-018 | 3 | 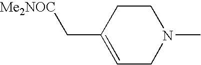 | N | CH₃ | CH₃ | CH₃ | 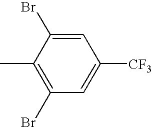 | amorphous |
| 1-019 | 2 | 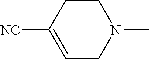 | N | CH₃ | CH₃ | CH₃ | 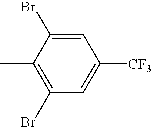 | amorphous |
| 1-020 | 2 | 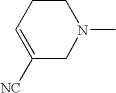 | N | CH₃ | CH₃ | CH₃ | 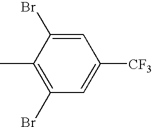 | amorphous |

*¹Com. No. = compound number, Ex. No. = example number, solvent for crystallization: EtOAc = ethyl acetate, IPE = diisopropylether
Analytical data of non-crystal compounds are described below.
1-007:
MS (ES, Pos): 500 (M + Na)⁺, 506 (M + Na + 2)⁺; NMR (300 MHz, CDCl₃) δ 1.01 (6 H, t, J=7.6 Hz), 1.93 (3 H, s), 1.95-2.20 (4 H, m), 2.37 (3 H, s), 2.48 (3 H, s), 2.51-2.64 (2 H, m), 3.61-3.72 (2 H, m), 4.12-4.24 (2 H, m), 6.77-6.88 (2 H, m).
1-008:
MS (ES, Pos): 486 (M + Na)⁺, 488 (M + Na + 2)⁺; NMR (300 MHz, CDCl₃) δ 1.02 (6 H, t, J=7.6 Hz), 2.05-2.30 (4 H, m), 2.44 (3 H, d, J=1.1 Hz), 2.49 (3 H, s), 2.51-2.67 (2 H, m), 3.67-3.78 (2 H, m), 4.18-4.30 (2 H, m), 6.60-6.63 (1 H, m), 6.82-6.89 (1 H, m), 7.35 (2 H, s).
1-015:
NMR (200 MHz, CDCl₃) δ 2.04 (3 H, s), 2.39 (3 H, s), 2.19-2.62 (2 H, m), 2.50 (3 H, s), 3.08-3.16 (2 H, m), 3.63-3.3.82 (2 H, m), 4.02-4.18 (2 H, m), 5.70-5.81 (1 H, m), 7.95 (1 H, d, J=0.8 Hz).
1-018:
NMR (200 MHz, CDCl₃) δ 2.04 (3 H, s), 2.38 (3 H, s), 2.30-2.60 (2 H, m), 2.48 (3 H, s), 2.98 (3 H, s), 3.04 (3 H, s), 3.11-3.20 (2 H, m), 3.61-3.3.80 (2 H, m), 4,02-4.15 (2 H, m), 5.56-5.68 (1 H, m), 7.95 (2 H, s).
1-019:
MS (ES, Pos): 568 (M + 1)⁺, 570 (M + 3)⁺, 572 (M + 5)⁺; NMR (300 MHz, CDCl₃) δ 2.06 (3 H, s), 2.36-2.42 (3 H, m), 2.49 (3 H, s), 2.58-2.68 (2 H, m), 3.68 (2 H, t, J=5.5 Hz), 4.16-4.25 (2 H, m), 6.71-6.79 (1 H, m), 7.93-7.99 (2 H, m).
1-020:
MS (ES, Pos): 568 (M + 1)⁺, 570 (M + 3)⁺, 572 (M + 5)⁺; NMR (300 MHz, CDCl₃) δ 2.06 (3 H, s), 2.36-2.42 (3 H, m), 2.50 (3 H, s), 2.53-2.62 (2 H, m), 3.68 (2 H, t, J=5.7 Hz), 4.15-4.24 (2 H, m), 6.78-6.87 (1 H, m), 7.93-7.99 (2 H, m).
*²The crystal was obtained after standing the compound purified with column chromatography.
*³1 HCl salt

### Test Example [CRF Receptor Binding Test]

Monkey amygdala membranes were used as a receptor preparation.

¹²⁵I-CRF was used as ¹²⁵I-labeled ligand.

Binding reaction using the ¹²⁵I-labeled ligand was carried out by the following method described in The Journal of Neuroscience, 7, 88 (1987).

Preparation of Receptor Membranes:

Monkey amygdala was homogenized in 50 mM Tris-HCl buffer (pH 7.0) containing 10 mM MgCl₂, 2 mM EDTA and centrifuged at 48,000×g for 20 min, and the precipitate was washed once with Tris-HCl buffer. The washed precipitate was suspended in 50 mM Tris-HCl buffer (pH 7.0) containing 10 mM MgCl₂, 2 mM EDTA, 0.1% bovine serum albumin and 100 kallikrein units/ml aprotinin, to obtain a membrane preparation.

CRF Receptor Binding Test:

The membrane preparation (0.3 mg protein/ml), ¹²⁵I-CRF (0.2 nM) and a test drug were reacted at 25° C. for 2 hours. After completion of the reaction, the reaction mixture was filtered by suction through a glass filter (GF/C) treated with 0.3% polyethylene imine, and the glass filter was washed three times with phosphate-buffered saline containing 0.01% Triton X-100. After the washing, the radioactivity of the filter paper was measured in a gamma counter.

The amount of ¹²⁵I-CRF bound when the reaction was carried out in the presence of 1 μM CRF was taken as the degree of nonspecific binding of ¹²⁵I-CRF, and the difference between the total degree of ¹²⁵I-CRF binding and the degree of nonspecific ¹²⁵I-CRF binding was taken as the degree of specific ¹²⁵I-CRF binding. An inhibition curve was obtained by reacting a definite concentration (0.2 nM) of ¹²⁵I-CRF with various concentrations of each test drug under the conditions described above. A concentration of the test drug at which binding of ¹²⁵I-CRF is inhibited by 50% (IC₅₀) was determined from the inhibition curve.

As a result, it was found that compounds 1-001, 1-002, 1-005, 1-006, 1-007, 1-008, 1-009, 1-010, 1-012, 1-014 can be exemplified as typical compounds having an IC₅₀ value of 100 nM or less.

### EFFECT OF THE INVENTION

According to the present invention, compounds having a high affinity for CRF receptors have been provided. These compounds are effective against diseases in which CRF is considered to be involved, such as depression, anxiety, Alzheimer's disease, Parkinson's disease, Huntington's chorea, eating disorder, hypertension, gastrointestinal diseases, drug dependence, cerebral infarction, cerebral ischemia, cerebral edema, cephalic external wound, inflammation, immunity-related diseases, alopecia, irritable bowel syndrome, sleep disorders, epilepsy, dermatitides, schizophrenia, pain, etc.

1. A pyrrolopyrimidine or pyrrolopyridine derivative substituted with tetrahydropyridine
represented by the following formula [I]: 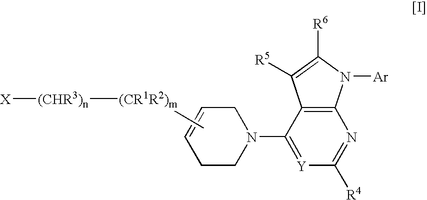
(wherein the tetrahydropyridine is represented by the following formula [II]: 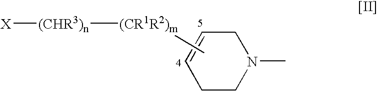
in which the tetrahydropyridine ring is substituted with a group represented by —(CR¹R²)ₘ—(CHR³)ₙ—X at the 4-position or 5-position of the tetrahydropyridine ring;
X is hydroxy, cyano, —CO₂R⁷ or —CONR^{7a}R^{7b};
Y is N or CR⁸;
with the proviso that when Y is CR⁸, then X is hydroxy;
R¹ is hydrogen, hydroxy, C₁₋₅alkyl, C₁₋₅alkoxy-C₁₋₅alkyl or hydroxy-C₁₋₅alkyl;
R² is hydrogen or C₁₋₅alkyl;
R³ is hydrogen, cyano, C₁₋₅alkyl, C₁₋₅alkoxy-C₁₋₅alkyl or hydroxy-C₁₋₅alkyl;
m is an integer selected from 0, 1, 2, 3, 4 and 5;
n is 0 or 1;
with the proviso that when X is hydroxy or —CONR^{7a}R^{7b}, and n is 0, then m is an integer selected from 1, 2, 3, 4 and 5;
R⁴ is hydrogen, halogen, C₁₋₅alkyl, C₃₋₈cycloalkyl, C₃₋₈cycloalkyl-C₁₋₅alkyl, hydroxy, C₁₋₅alkoxy, C₃₋₈cycloalkyloxy or —N(R⁹)R¹⁰;
R⁵ and R⁶ are the same or different, and independently are hydrogen, halogen, C₁₋₅alkyl, C₃₋₈cycloalkyl, C₃₋₈cycloalkyl-C₁₋₅alkyl, hydroxy, C₁₋₅alkoxy, C₃₋₈cycloalkyloxy, —N(R¹¹)R¹², —CO₂R¹³, cyano, nitro, C₁₋₅alkylthio, trifluoromethyl or trifluoromethoxy; or R⁵ and R⁶ are taken together to form —CH₂—CH₂—CH₂—CH₂— or —CH═CH—CH═CH—;
with the proviso that when R⁵ and R⁶ are taken together to form —CH₂—CH₂—CH₂—CH₂—, then X is hydroxy;
R⁷ is hydrogen or C₁₋₅alkyl;
R^{7a} and R^{7b} are the same or different, and independently hydrogen or C₁₋₅alkyl;
R⁸ is hydrogen, C₁₋₅alkyl, halogen, cyano or —CO₂R¹⁴;
R⁹ and R¹⁰ are the same or different, and independently are hydrogen, C₁₋₅alkyl, C₃₋₈cycloalkyl or C₃₋₈cycloalkyl-C₁₋₅alkyl;
R¹¹ and R¹² are the same or different, and independently are hydrogen, C₁₋₅alkyl, C₃₋₈cycloalkyl or C₃₋₈cycloalkyl-C₁₋₅alkyl;
R¹³ is hydrogen or C₁₋₅alkyl;
R¹⁴ is hydrogen or C₁₋₅alkyl;
Ar is aryl or heteroaryl which aryl or heteroaryl is unsubstituted or substituted with 1 or more substituents, which are the same or different, selected from the group consisting of halogen, C₁₋₅alkyl, C₃₋₈cycloalkyl, C₂₋₅alkenyl, C₂₋₅alkynyl, C₁₋₅alkoxy, C₁₋₅alkylthio, C₁₋₅alkylsulfinyl, C₁₋₅alkylsulfonyl, cyano, nitro, hydroxy, —CO₂R¹⁵, —C(═O)R¹⁶, —CONR¹⁷R¹⁸, —C(═O)R¹⁹, —NR²⁰CO₂R²¹, —S(O)ᵣNR²²R²³, trifluoromethyl, trifluoromethoxy, difluoromethoxy, fluoromethoxy, methylenedioxy, ethylenedioxy and —N(R²⁴)R²⁵;
R¹⁵ is hydrogen, C₁₋₅alkyl, C₃₋₈cycloalkyl or C₃₋₈cycloalkyl-C₁₋₅alkyl;
R¹⁶ is hydrogen or C₁₋₅alkyl;
R¹⁷ and R¹⁸ are the same or different, and independently are hydrogen, C₁₋₅alkyl, C₃₋₈cycloalkyl or C₃₋₈cycloalkyl-C₁₋₅alkyl;
R¹⁹ is hydrogen or C₁₋₅alkyl;
R²⁰ is hydrogen or C₁₋₅alkyl;
R²¹ is hydrogen or C₁₋₅alkyl;
R²² and R²³ are the same or different, and independently are hydrogen, C₁₋₅alkyl, C₃₋₈cycloalkyl or C₃₋₈cycloalkyl-C₁₋₅alkyl;
R²⁴ and R²⁵ are the same or different, and independently are hydrogen, C₁₋₅alkyl, C₃₋₈cycloalkyl or C₃₋₈cycloalkyl-C₁₋₅alkyl;
r is 1 or 2), individual isomers thereof, racemic or non-racemic mixtures of isomers thereof or N-oxide thereof, or pharmaceutically acceptable salts and hydrates thereof.

2. A pyrrolopyrimidine or pyrrolopyridine derivative substituted with tetrahydropyridine represented by the following formula [I]: 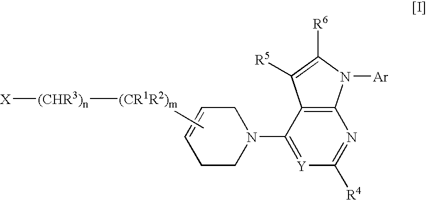
(wherein the tetrahydropyridine is represented by the following formula [II]: 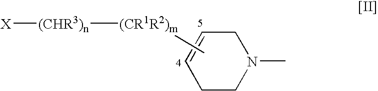
in which the tetrahydropyridine ring is substituted with a group represented by —(CR¹R²)ₘ—(CHR³)ₙ—X at the 4-position or 5-position of the tetrahydropyridine ring;
X is hydroxy, cyano or —CO₂R⁷;
Y is N or CR⁸;
with the proviso that when Y is CR⁸, then X is hydroxy;
R¹ is hydrogen, hydroxy, C₁₋₅alkyl, C₁₋₅alkoxy-C₁₋₅alkyl or hydroxy-C₁₋₅alkyl;
R² is hydrogen or C₁₋₅alkyl;
R³ is hydrogen, cyano, C₁₋₅alkyl, C₁₋₅alkoxy-C₁₋₅alkyl or hydroxy-C₁₋₅alkyl;
m is an integer selected from 0, 1, 2, 3, 4 and 5;
n is 0 or 1;
with the proviso that when X is hydroxy, and n is 0, then m is an integer selected from 1, 2, 3, 4 and 5;
R⁴ is hydrogen, C₁₋₅alkyl, C₃₋₈cycloalkyl, C₃₋₈cycloalkyl-C₁₋₅alkyl, hydroxy, C₁₋₅alkoxy, C₃₋₈cycloalkyloxy or —N(R⁹)R¹⁰;
R⁵ and R⁶ are the same or different, and independently are hydrogen, halogen, C₁₋₅alkyl, C₃₋₈cycloalkyl, C₃₋₈cycloalkyl-C₁₋₅alkyl, hydroxy, C₁₋₅alkoxy, C₃₋₈cycloalkyloxy, —N(R¹¹)R¹², —CO₂R¹³, cyano, nitro, C₁₋₅alkylthio, trifluoromethyl or trifluoromethoxy; or R⁵ and R⁶ are taken together to form —CH₂—CH₂—CH₂—CH₂— or —CH═CH—CH═CH—;
with the proviso that when R⁵ and R⁶ are taken together to form —CH₂—CH₂—CH₂—CH₂—, then X is hydroxy;
R⁷ is hydrogen or C₁₋₅alkyl;
R⁸ is hydrogen, C₁₋₅alkyl, halogen, cyano or —CO₂R¹⁴;
R⁹ and R¹⁰ are the same or different, and independently are hydrogen, C₁₋₅alkyl, C₃₋₈cycloalkyl or C₃₋₈cycloalkyl-C₁₋₅alkyl;
R¹¹ and R¹² are the same or different, and independently are hydrogen, C₁₋₅alkyl, C₃₋₈cycloalkyl or C₃₋₈cycloalkyl-C₁₋₅alkyl;
R¹³ is hydrogen or C₁₋₅alkyl;
R¹⁴ is hydrogen or C₁₋₅alkyl;
Ar is aryl or heteroaryl which aryl or heteroaryl is unsubstituted or substituted with 1 or more substituents, which are the same or different, selected from the group consisting of halogen, C₁₋₅alkyl, C₃₋₈cycloalkyl, C₂₋₅alkenyl, C₂₋₅alkynyl, C₁₋₅alkoxy, C₁₋₅alkylthio, C₁₋₅alkylsulfinyl, C₁₋₅alkylsulfonyl, cyano, nitro, hydroxy, —CO₂R¹⁵, —C(═O)R¹⁶, —CONR¹⁷R¹⁸, —OC(═O)R¹⁹, —NR²⁰CO₂R²¹, —S(O)ᵣNR²²R²³, trifluoromethyl, trifluoromethoxy, difluoromethoxy, fluoromethoxy, methylenedioxy, ethylenedioxy and —N(R²⁴)R²⁵;
R¹⁵ is hydrogen, C₁₋₅alkyl, C₃₋₈cycloalkyl or C₃₋₈cycloalkyl-C₁₋₅alkyl;
R¹⁶ is hydrogen or C₁₋₅alkyl;
R¹⁷ and R¹⁸ are the same or different, and independently are hydrogen, C₁₋₅alkyl, C₃₋₈cycloalkyl or C₃₋₈cycloalkyl-C₁₋₅alkyl;
R¹⁹ is hydrogen or C₁₋₅alkyl;
R²⁰ is hydrogen or C₁₋₅alkyl;
R²¹ is hydrogen or C₁₋₅alkyl;
R²² and R²³ are the same or different, and independently are hydrogen, C₁₋₅alkyl, C₃₋₈cycloalkyl or C₃₋₈cycloalkyl-C₁₋₅alkyl;
R²⁴ and R²⁵ are the same or different, and independently are hydrogen, C₁₋₅alkyl, C₃₋₈cycloalkyl or C₃₋₈cycloalkyl-C₁₋₅alkyl;
r is 1 or 2), individual isomers thereof, racemic or non-racemic mixtures of isomers thereof or N-oxide thereof, or pharmaceutically acceptable salts and hydrates thereof.

3. The pyrrolopyrimidine derivative substituted with the tetrahydropyridine according to claim 2 represented by formula [I], wherein Y is N; X, m, n, R¹, R², R³, R⁴, R⁵, R⁶ and Ar are as defined in claim 2; individual isomers thereof or racemic or non-racemic mixtures of isomers thereof, or pharmaceutically acceptable salts and hydrates thereof.

4. The pyrrolopyrimidine derivative substituted with the tetrahydropyridine according to claim 2 represented by formula [I], wherein Y is N; X is hydroxy; m is an integer selected from 1, 2, 3, 4 and 5; n is 0; R¹ and R² are hydrogen; R⁴, R⁵, R⁶ and Ar are as defined in claim 2; individual isomers thereof or racemic or non-racemic mixtures of isomers thereof, or pharmaceutically acceptable salts and hydrates thereof.

5. The pyrrolopyrimidine derivative substituted with the tetrahydropyridine according to claim 2 represented by formula [I], wherein Y is N; X is hydroxy; m is an integer selected from 1, 2 and 3; n is 0; R¹ and R² are hydrogen; R⁴ is C₁₋₅alkyl; R⁵ and R⁶ are the same or different, and independently are hydrogen or C₁₋₅alkyl; Ar is phenyl which phenyl is substituted with two or three substituents, which are the same or different, selected from the group consisting of halogen, C₁₋₃alkyl, C₁₋₃alkoxy, C₁₋₃alkylthio, trifluoromethyl, trifluoromethoxy and —N(R²⁴)R²⁵ (wherein R²⁴ and R²⁵ are the same or different, and independently are hydrogen or C₁₋₃alkyl); individual isomers thereof or racemic or non-racemic mixtures of isomers thereof, or pharmaceutically acceptable salts and hydrates thereof.

6. The pyrrolopyrimidine derivative substituted with the tetrahydropyridine according to claim 2 represented by formula [I], wherein Y is N; X is cyano; R¹, R² and R³ are hydrogen; m, n, R⁴, R⁵, R⁶ and Ar are as defined in claim 2; individual isomers thereof or racemic or non-racemic mixtures of isomers thereof, or pharmaceutically acceptable salts and hydrates thereof.

7. The pyrrolopyrimidine derivative substituted with the tetrahydropyridine according to claim 2 represented by formula [I], wherein Y is N; X is cyano; m is 0 or 1; n is 0; R¹ and R² are hydrogen; R⁴ is C₁₋₅alkyl; R⁵ and R⁶ are the same or different, and independently are hydrogen or C₁₋₅alkyl; Ar is phenyl which phenyl is substituted with two or three substituents, which are the same or different, selected from the group consisting of halogen, C₁₋₃alkyl, C₁₋₃alkoxy, C₁₋₃alkylthio, trifluoromethyl, trifluoromethoxy and —N(R²⁴)R²⁵ (wherein R²⁴ and R²⁵ are the same or different, and independently are hydrogen or C₁₋₃alkyl); individual isomers thereof or racemic or non-racemic mixtures of isomers thereof, or pharmaceutically acceptable salts and hydrates thereof.

8. The pyrrolopyridine derivative substituted with the tetrahydropyridine according to claim 2 represented by formula [I], wherein Y is CR⁸; X is hydroxy; m, n, R¹, R², R³, R⁴, R⁵, R⁶, R⁸ and Ar are as defined in claim 2; individual isomers thereof or racemic or non-racemic mixtures of isomers thereof, or pharmaceutically acceptable salts and hydrates thereof.

9. The pyrrolopyridine derivative substituted with the tetrahydropyridine according to claim 2 represented by formula [I], wherein Y is CH; X is hydroxy; m is an integer selected from 1, 2, 3, 4 and 5; n is 0; R¹ and R² are hydrogen; R⁴, R⁵, R⁶ and Ar are as defined in claim 2; individual isomers thereof or racemic or non-racemic mixtures of isomers thereof, or pharmaceutically acceptable salts and hydrates thereof.

10. The pyrrolopyridine derivative substituted with the tetrahydropyridine according to claim 2 represented by formula [I], wherein Y is CH; X is hydroxy; m is an integer selected from 1, 2 and 3; n is 0; R¹ and R² are hydrogen; R⁴ is C₁₋₅alkyl; R⁵ and R⁶ are the same or different, and independently are hydrogen or C₁₋₅alkyl; Ar is phenyl which phenyl is substituted with two or three substituents, which are the same or different, selected from the group consisting of halogen, C₁₋₃alkyl, C₁₋₃alkoxy, C₁₋₃alkylthio, trifluoromethyl, trifluoromethoxy and —N(R²⁴)R²⁵ (wherein R²⁴ and R²⁵ are the same or different, and independently are hydrogen or C₁₋₃alkyl); individual isomers thereof or racemic or non-racemic mixtures of isomers thereof, or pharmaceutically acceptable salts and hydrates thereof.

11. An antagonist for CRF receptors, comprising a pyrrolopyrimidine or pyrrolopyridine derivative substituted with tetrahydropyridine, a pharmaceutically acceptable salt thereof or its hydrate according to claim 1, as an active ingredient.

12. Use of a pyrrolopyrimidine or pyrrolopyridine derivative substituted with tetrahydropyridine, a pharmaceutically acceptable salt thereof or its hydrate according to claim 1, for the manufacture of an antagonist for CRF receptors.
